(19) United States
(12) Patent Application Publication
(10) Pub. No.: US 2018/0059129 A1
(43) Pub. Date: Mar. 1, 2018

(54) AUTOMATED URINALYSIS

(71) Applicant: Rajan Dewar, Northborough, MA (US)

(72) Inventor: Rajan Dewar, Northborough, MA (US)

(21) Appl. No.: 15/685,047

(22) Filed: Aug. 24, 2017

Related U.S. Application Data
(60) Provisional application No. 62/379,430, filed on Aug. 25, 2016.

(51) Int. Cl. G01N 35/00; G01N 33/487

(52) U.S. Cl. CPC G01N 35/00871; A61B 5/201; G01N 33/48764

(57) ABSTRACT

A system for automated urinalysis includes an in-line sensor disposed in a conduit between a sanitation device such as a toilet and a standard waste treatment system. The sensor may include a matrix of reagent strips. An image of the exposed matrix of reagent strips is captured with a CCD camera. The exposed matrix of reagent strips may be illuminated by an LED in order to facilitate image acquisition. A representation of the image is transmitted to an analysis program. Results of the analysis may be transmitted to a mobile device such as a smart phone.

### CROSS-REFERENCE TO RELATED APPLICATIONS

This application claims priority to U.S. Provisional Patent Application 62/379,430, titled AUTOMATED URINALYSIS AND RESULTING, filed 25 Aug. 2016, which is incorporated by reference.

### BACKGROUND

Biological specimens are used to diagnose and monitor a variety of medical conditions. Examples of biological specimens may include but are not limited to peripheral blood, urine, and other body fluids, e.g. pleural or pericardial. On average, a human being excretes 750-1500 ml of fresh urine per day with a pH value of around 7.2. The urinary content may vary considerably depending on factors including but not limited to hydration. Urinary content may indicate disease states including but not limited to diabetes, systemic illnesses, kidney disorders, and local infections, e.g. urinary tract infections. Urine may also be used to detect drug use and drug levels, e.g. for therapeutic analysis or as an indication of drug abuse. Typically, a urine sample is collected in a sample container, the container is transported to a test lab, and the urine sample is removed from the container for testing.

### SUMMARY

All examples, aspects and features mentioned in this document can be combined in any technically possible way. Unless otherwise stated, use of the word “substantially” may be construed to include a precise relationship, condition, arrangement, orientation, and/or other characteristic, and deviations thereof as understood by one of ordinary skill in the art, to the extent that such deviations do not materially affect the disclosed methods and systems. Throughout the entirety of the present disclosure, use of the articles “a” or “an” to modify a noun may be understood to be used for convenience and to include one, or more than one of the modified noun, unless otherwise specifically stated.

In accordance with an aspect an apparatus comprises: a sensor disposed in a conduit between a sanitation device and waste treatment, the sensor comprising a sensing element that detects characteristics of urine, and a transmitter circuit that transmits the detected characteristics of the urine. In some implementations the sensing element comprises at least one reagent strip. In some implementations the sensing element comprises a matrix of reagent strips. In some implementations the transmitter circuit comprises a wireless transmitter. Some implementations further comprise an analysis computer that receives the detected characteristics of the urine. Some implementations further comprise an analysis program that runs on the analysis computer, the analysis program generating an analysis of the detected characteristics of the urine. In some implementations the analysis computer sends the analysis of the detected characteristics of the urine to a mobile device. In some implementations the sensor comprises a light emitting diode that illuminates the sensing element. In some implementations the sensor comprises an imaging chip that captures an image of the sensing element. In some implementations the sensing element comprises a reagent chip disposed on a strip of reagent chips, and wherein the sensor comprises a feeder reel of unexpended ones of the reagent chips and a collection reel of expended ones of the reagent chips. Some implementations comprise at least one pulley that guides the strip of reagent chips. Some implementations comprise an imaging device that detects at least one color of one of the expended reagent chips.

In accordance with an aspect a method comprises: performing automated urinalysis, comprising the steps of: positioning an unexpended reagent strip for exposure to urine flowing from a sanitation device to waste treatment; exposing the unexpended reagent strip to the urine, thereby expending the reagent strip; detecting characteristics of the urine sample from the expended reagent strip; and transmitting the detected characteristics of the urine. Some implementations comprise detecting characteristics of the urine sample from the expended reagent strip comprises capturing an image of the expended reagent strip. In some implementations transmitting the detected characteristics of the urine comprises transmitting a representation of the captured image to an analysis computer. Some implementations comprise analyzing urinary content based on the transmitted representation of the captured image. Some implementations comprise transmitting an analysis of urinary content to a mobile device.

In accordance with an aspect an apparatus comprises: a conduit comprising an inlet and an outlet, the inlet fluidically connected with a sanitation device, the outlet fluidically connected with waste treatment; a strip of reagent chips; guides that position a selected one of the reagent chips in the conduit, whereby the selected reagent chip is exposed to urine and expended; a motor that advances the strip such that the expended reagent chip is repositioned outside of the conduit; an imaging device that captures a representation of an image of the repositioned expended reagent chip; and a transmitter that transmits the representation to an analysis computer that analyzes urinary content based on the representation. Some implementations comprise a feeder reel of unexpended ones of the reagent chips and a collection reel of expended ones of the reagent chips. Some implementations comprise a mobile device application that receives an analysis of urinary content from the analysis computer.

### BRIEF DESCRIPTION OF THE DRAWINGS

FIG. 1 is a block diagram of a system for automated urinalysis.

FIG. 2 illustrates the sensor subsystem of FIG. 1 in greater detail.

FIG. 3 illustrates another implementation of the sensor subsystem of FIG. 1 in greater detail.

FIG. 4 is a flow diagram of a process for automated urinalysis.

### DETAILED DESCRIPTION

The detailed description set forth below will enable those of ordinary skill in the art to practice the invention. Upon reading the detailed description in light of the accompanying figures, those of ordinary skill in the art will understand the concepts of the invention and recognize further variations and implementations of these concepts. It should be understood that these further variations and implementations fall within the scope of the disclosure.

Some aspects, features and implementations described herein may include machines such as computer devices, electronic components, optical components, and processes such as computer-implemented steps. It will be apparent to those of ordinary skill in the art that the computer-implemented steps may be stored as computer-executable instructions on a non-transitory computer-readable medium. Furthermore, it will be understood by those of ordinary skill in the art that the computer-executable instructions may be executed on a variety of tangible processor devices. For ease of exposition, not every step, device or component that may be part of a computer or data storage system is described herein. Those of ordinary skill in the art will recognize such steps, devices and components in view of the teachings of the present disclosure and the knowledge generally available to those of ordinary skill in the art. The corresponding machines and processes are therefore enabled and within the scope of the disclosure.

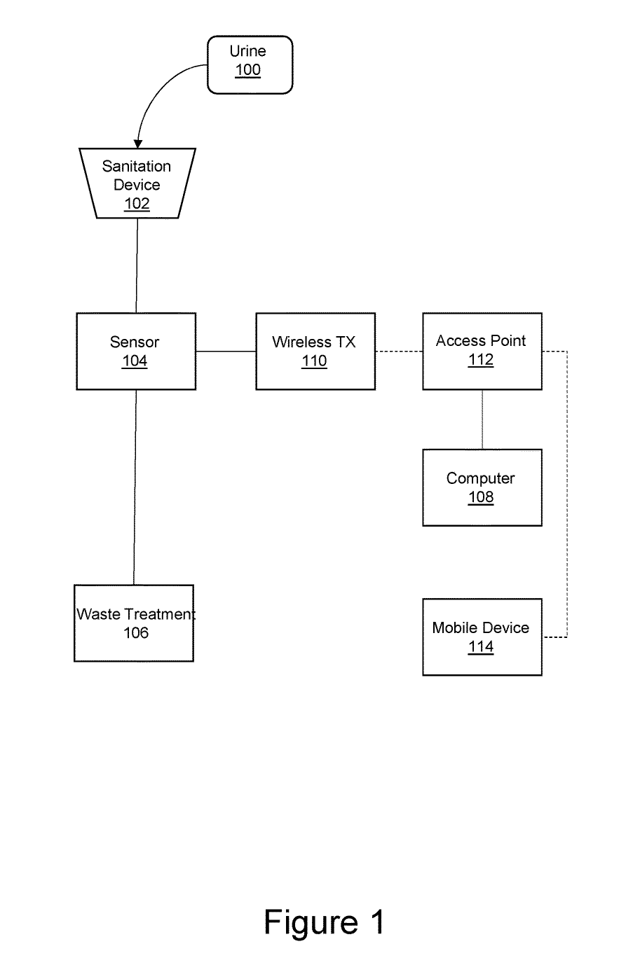

FIG. 1 illustrates a system for automated urinalysis. The plumbing of a conventional sanitation system may be modified or retrofitted for automated urinalysis. One of the drawbacks of non-automated urinalysis is that urine specimens are manually collected and processed. Another drawback of existing urinalysis techniques is that there may be a significant delay between sample collection and delivery of test results. Some implementations described herein may help to overcome those drawbacks, although possible advantages should not be viewed as limitations of the inventive concepts.

In the illustrated automated urinalysis system, urine 100 is deposited directly into a sanitation device 102. More specifically, a human being urinates directly into a toilet, urinal, or other type of sanitation device. At least some of the urine 100 is provided to a sensor 104 that senses characteristics of the urine 100. The sensed characteristics may be indicative of, but are not limited to, pH, specific gravity, protein, glucose, ketones, leucocyte esterase, nitrite, bilirubin, and urobilinogen. The urine 100 that was processed by the sensor may be directed to conventional waste treatment 106, e.g. and without limitation a municipal sewage system. The urinary content characteristics detected by the sensor 104 are transmitted to an analysis computer 108. For example and without limitation, the urinary content characteristics may be transmitted by a wireless transmitter 110 to the analysis computer 108 via a wireless access point 112. The analysis computer 108 includes an analysis program that includes algorithms for processing the received urinary content characteristics, e.g. and without limitation to detect indications of medical conditions, drug levels, or drug use. An output of the analysis program may be sent to a user terminal such as a mobile device 114 via the access point 112. An application running on the mobile device may receive the output, display the output, organize the output, and provide further analysis. Thus, a urine sample may be analyzed in real time (without delays associated with transport and temporary storage of samples awaiting analysis) without being manually handled.

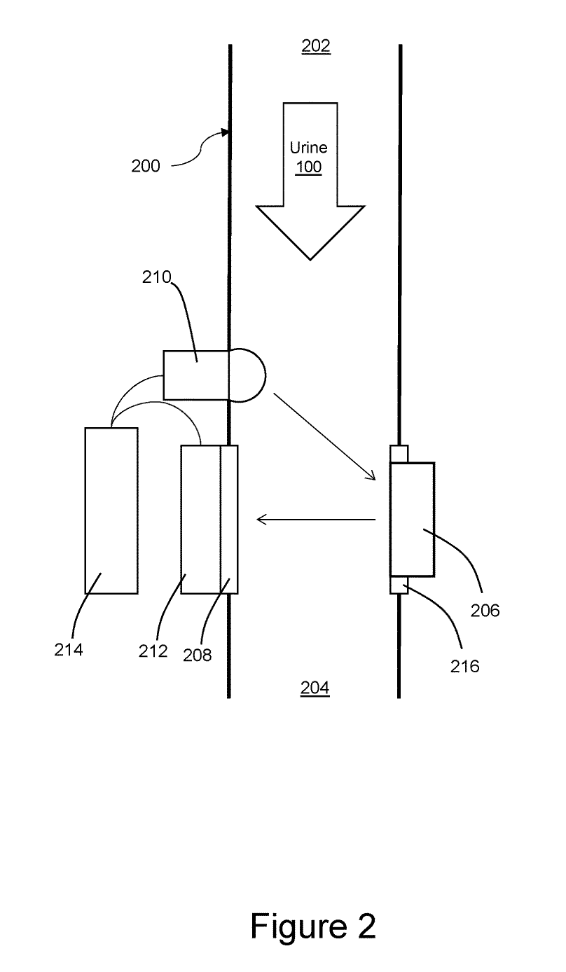

FIG. 2 illustrates an implementation of the sensor (104, FIG. 1) in greater detail. A conduit 200 is connected between the receptacle (102, FIG. 1) and waste treatment (106, FIG. 1). The urine 100 is introduced to the conduit via an inlet 202, flows through the sensor, and exits the conduit via an outlet 204. Although the illustration may suggest a vertical conduit orientation, any of a wide variety of orientations are possible. The sensor may include a reagent chip 206, a CCD (charge coupled device) imaging chip 208, an LED (light emitting diode) 210, an RF (radio frequency) transmitter 212, and a power source 214. The reagent chip 206 may include a matrix of reagent strips, e.g. and without limitation a 3×4 matrix of individual paper reagent strips. The cross-sectional diameter of the fluid conduit 200 may be selected to restrict urine flow such that the reagent chip 206 is exposed to the urine 100 produced during a single urination event for 60 seconds, or some other amount of time sufficient to enable the chemical reactions between the reagent chip and the urine. The reagent chip 206 indicates urinary content characteristics via color changes that result from chemical reactions between the reagent strips and the urine. The CCD imaging chip 208 obtains an image of the reagent chip 206 following exposure to the urine 100, thereby recording the color and/or changes resulting from the chemical reactions. The output of the CCD imaging chip 208, e.g. an image or representation thereof, is transmitted by the RF transmitter 212. Because the interior volume of the fluid conduit 200 may be shielded from ambient light, the LED 210 is used to illuminate the reagent chip 206 so that an image of the colors can be obtained by the CCD imaging chip 208. The characteristics of the LED may be selected such that the generated light does not interfere with the ability of the system to accurately detect and analyze colors and color changes of the reagent strips. The reagent chip may be mounted in a port 216 formed through the wall of the conduit. The power source 214 may include a battery, for example and without limitation. The color change of the reagent chip is interpreted by the automated image analysis program in the computer (108, FIG. 1). The expended reagent chip is removed from the port 216 and replaced with an unused reagent chip in order to prepare the sensor for another test.

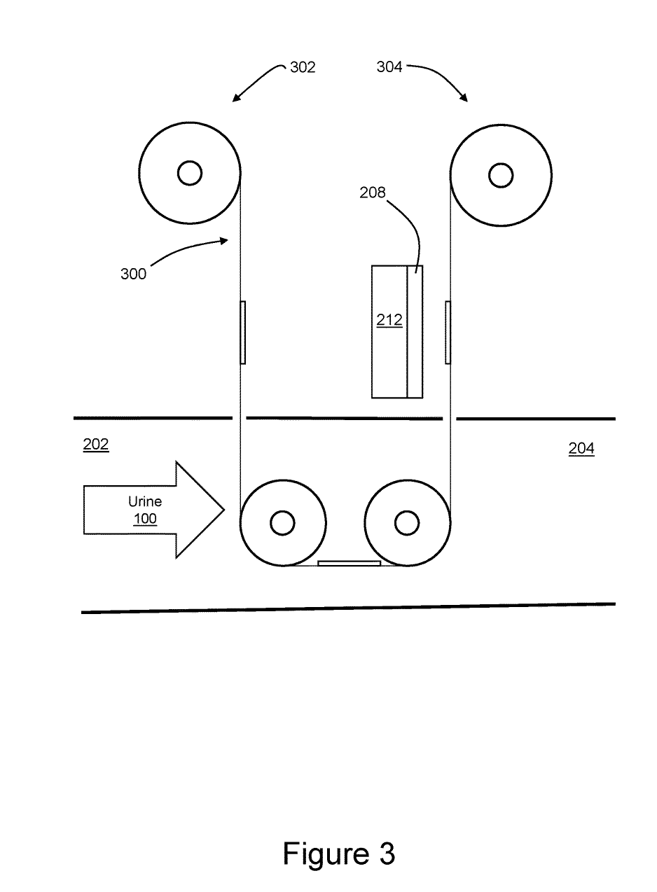

FIG. 3 illustrates another implementation of the sensor subsystem of FIG. 1 in greater detail. In this implementation reagent chips are attached to a strip 300 that can be wound around reels. In the illustrated example the strip 300 is unwound from a feeder reel 302 and wound onto a collection reel 304. Pulleys are used to position a reagent chip in place for exposure to the urine 100. The pulleys are located within a conduit through which the urine flows from an inlet to an outlet. Unexpended reagent chips are dispensed from the feeder reel through a first opening in the conduit. For example, an unexpended reagent chip may be positioned between the pulleys at a level that facilitates exposure to the urine for 60 seconds, or some other amount of time sufficient to enable the chemical reactions between the reagent chip and the urine. Following exposure, the reagent chip is moved into position relative to the CCD imaging chip 208. An image or representation thereof is transmitted by the RF transmitter 212 as already described above. The positioning of the pulleys and spacing between reagent chips may be such that a reagent chip is aligned with the CCD imaging chip when the adjacent reagent chip is positioned between the pulleys for exposure to urine. Expended reagent chips are wound on to the collection reel. The reels may be replaced when all of the reagent chips on the strip have been expended. A wide variety of mechanisms may be used to advance the strip, including but not limited to a stepper motor.

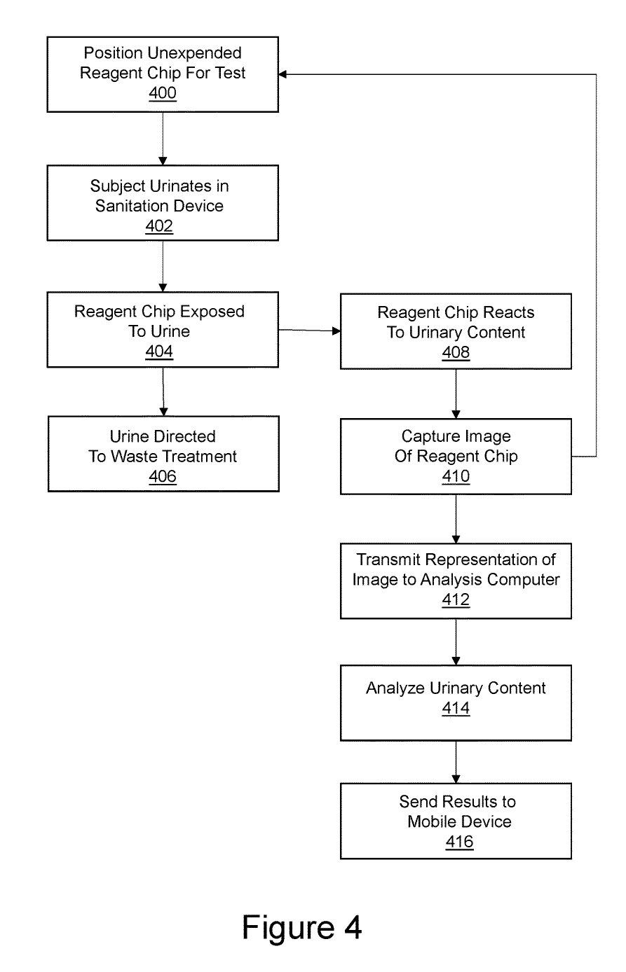

FIG. 4 is a flow diagram of a process for automated urinalysis. An unexpended reagent chip is positioned for a test as indicated in block 400. This may include the positioning of a single reagent chip in a conduit or container, or automated positioning of a reagent chip on a strip. The subject urinates in the sanitation device as indicated in block 402. The reagent chip is exposed to urine as indicated in block 404. As indicated above, the exposure time may be controlled. The urine is directed to waste treatment as indicated in block 406. For example, the urine may be directed to a standard waste treatment system such as a municipal sewer via standard plumbing. The reagent chip reacts to the urinary content as indicated in block 408. For example and without limitation, the reagent chip may react to the urinary content by changing color. An image of the reacted reagent chip is captured as indicated in block 410. Another unexpended reagent chip may be positioned for a test as indicated in block 400. A representation of the captured image is transmitted to the analysis computer as indicated in block 412. For example, a numerical representation of the color(s) captured in the image may be transmitted. The transmission could be wireless or wireline. The analysis computer processes the representation in order to analyze the urinary content as indicated in block 414. The results may be sent to a mobile device, and/or any other selected devices, as indicated in block 416. A mobile device application may receive the results, display the results, organize the results, and provide further analysis.

A number of features, aspects, embodiments and implementations have been described. Nevertheless, it will be understood that a wide variety of modifications and combinations may be made without departing from the scope of the inventive concepts described herein. Accordingly, those modifications and combinations are within the scope of the following claims.

1. An apparatus comprising:
a sensor disposed in a conduit between a sanitation device and waste treatment, the sensor comprising a sensing element that detects characteristics of urine, and a transmitter circuit that transmits the detected characteristics of the urine.

2. The apparatus of claim 1 wherein the sensing element comprises at least one reagent strip.

3. The apparatus of claim 1 wherein the sensing element comprises a matrix of reagent strips.

4. The apparatus of claim 1 wherein the transmitter circuit comprises a wireless transmitter.

5. The apparatus of claim 1 further comprising an analysis computer that receives the detected characteristics of the urine.

6. The apparatus of claim 5 further comprising an analysis program that runs on the analysis computer, the analysis program generating an analysis of the detected characteristics of the urine.

7. The apparatus of claim 6 wherein the analysis computer sends the analysis of the detected characteristics of the urine to a mobile device.

8. The apparatus of claim 1 wherein the sensor comprises a light emitting diode that illuminates the sensing element.

9. The apparatus of claim 1 wherein the sensor comprises an imaging chip that captures an image of the sensing element.

10. The apparatus of claim 1 wherein the sensing element comprises a reagent chip disposed on a strip of reagent chips, and wherein the sensor comprises a feeder reel of unexpended ones of the reagent chips and a collection reel of expended ones of the reagent chips.

11. The apparatus of claim 10 comprising at least one pulley that guides the strip of reagent chips.

12. The apparatus of claim 11 comprising an imaging device that detects at least one color of one of the expended reagent chips.

13. A method comprising:
performing automated urinalysis, comprising the steps of:
positioning an unexpended reagent strip for exposure to urine flowing from a sanitation device to waste treatment;
exposing the unexpended reagent strip to the urine, thereby expending the reagent strip;
detecting characteristics of the urine sample from the expended reagent strip; and
transmitting the detected characteristics of the urine.

14. The method of claim 13 wherein detecting characteristics of the urine sample from the expended reagent strip comprises capturing an image of the expended reagent strip.

15. The method of claim 14 wherein transmitting the detected characteristics of the urine comprises transmitting a representation of the captured image to an analysis computer.

16. The method of claim 15 comprising analyzing urinary content based on the transmitted representation of the captured image.

17. The method of claim 16 comprising transmitting an analysis of urinary content to a mobile device.

18. An apparatus comprising:
a conduit comprising an inlet and an outlet, the inlet fluidically connected with a sanitation device, the outlet fluidically connected with waste treatment;
a strip of reagent chips;
guides that position a selected one of the reagent chips in the conduit, whereby the selected reagent chip is exposed to urine and expended;
a motor that advances the strip such that the expended reagent chip is repositioned outside of the conduit;
an imaging device that captures a representation of an image of the repositioned expended reagent chip; and
a transmitter that transmits the representation to an analysis computer that analyzes urinary content based on the representation.

19. The apparatus of claim 18 comprising a feeder reel of unexpended ones of the reagent chips and a collection reel of expended ones of the reagent chips.

20. The apparatus of claim 19 comprising a mobile device application that receives an analysis of urinary content from the analysis computer.
